(19) United States
(12) Patent Application Publication
(10) Pub. No.: US 2025/0076125 A1
(43) Pub. Date: Mar. 6, 2025

(54) Urine Temperature Monitor

(71) Applicant: Michael Heisler, Abingdon, VA (US)

(72) Inventor: Michael Heisler, Abingdon, VA (US)

(21) Appl. No.: 18/238,702

(22) Filed: Aug. 28, 2023

(51) Int. Cl. G01K 1/024; B01L 3/00; B01L 7/00

(52) U.S. Cl. CPC G01K 1/024; B01L 3/508; B01L 7/00; B01L 2300/0663; B01L 2300/1805; G08B 6/00

(57) ABSTRACT

A device which monitors and notifies the user of urine temperature. The device provides the user with an acceptable range for urine analysis, allowing the user to know if the urine temperature is either too hot or too cold. The user is notified if the temperature is out of the acceptable range.

### BACKGROUND OF THE INVENTION

The present subject matter is designed to allow users to withhold medical information during urine analysis. For over 25 years there has been an electronic device on the market that allows the user to electronically maintain proper urine temperature using digital electronics.

Even though there are many devices on the market that attempt to maintain proper urine temperature with electronic heating pads or organic heat pads or other sources.

All units on the market use LCD temperature strips that are stuck to the fluid container for the user to determine the actual temperature or a local digital readout display located on the fluid container. No product has the ability to remotely view urine temperature.

The purpose of the present subject matter is to allow the user to remotely monitor the urine temperature without having to look at the fluid container.

### SUMMARY OF THE INVENTION

In the following description, numerous specific details are set forth to clearly describe various specific embodiments disclosed herein. One skilled in the art, however, will understand that the presently claimed invention may be practiced without all of the specific details discussed below. In other instances, well known features have not been described so as not to obscure the invention.

As described herein, the term “pivotally connected” shall be used to describe a situation wherein two or more identified objects are joined together in a manner that allows one or both of the objects to pivot, and/or rotate about or in relation to the other object in either a horizontal or vertical manner.

As described herein, the term “removably coupled” and derivatives thereof shall be used to describe a situation wherein two or more objects are joined together in a non-permanent manner so as to allow the same objects to be repeatedly joined and separated.

Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings.

The present subject is designed to allow the user to remotely monitor and notify them of their urine temperature. The present subject matter uses of Bluetooth, wireless, Wi-Fi, or any type of technology that allows a user to monitor or be notified of the urine temperature and or battery status from a remote location.

The present subject matter gives indications to the user about the appropriate temperature ranges for their urine. In some embodiments, the device also provides the user with notifications regarding battery life remaining.

I'm some embodiments, the temperature indicator provides feedback through an app in which the user receives a color-coded range of temperatures and where the temperature of their urine falls. The temperature gauge indicates red when the temperature of the urine is too hot for the upcoming analysis. The temperature gauge indicates yellow when the temperature of the is too low for the urine analysis. The temperature gauge indicates green when the temperature of the urine is within the acceptable temperature parameters for urine analysis.

The display is shown as a semi-circle with that semi-circle divided into the three different colored sections wherein yellow is on the left portion of the semi-circle, green is in the middle section of the semi-circle, and red is on the right portion of the semi-circle. The application also provides the exact urine temperature on the user's phone.

In some embodiments, the application will also send vibrating signals to the user through their phone to alert if the temperature is too high or too low. The present subject matter features a rapid vibration when the temperature is too low and a slow vibration when the battery life is low.

The present subject matter also features a confidential mode wherein any communication between the device and the user's phone is encrypted. The user also has the ability to activate or deactivate the heating element of the device while still being able to monitor the temperature. In some embodiments, the heating element comprises of a heating pad.

In some embodiments, air activated heating pads may be used to heat the urine.

While several illustrative embodiments of the invention have been shown and described, numerous variations and alternative embodiments will occur to those skilled in the art. Such variations and alternative embodiments are contemplated, and can be made without departing from the scope of the invention as defined in the appended claims.

### BRIEF DESCRIPTION OF DRAWINGS

FIG. 1 shows a screenshot of the application wherein the user can activate stealth mode, show battery status, and various vibration settings.

FIG. 2 shows a temperature display to indicate when the urine temperature is acceptable for urine analysis.

FIG. 3 shows a front view of the urine temperature monitor.

FIG. 4 shows a front view of the fluid bag.

FIG. 5 shows a side view of the fluid bag with a heating pad.

FIG. 6 shows a top view of the Bluetooth module and heating pad switch.

FIG. 7 shows a bottom view of the Bluetooth module and heating pad switch.

FIG. 8 shows a cutaway view of the Bluetooth module and heating pad switch.

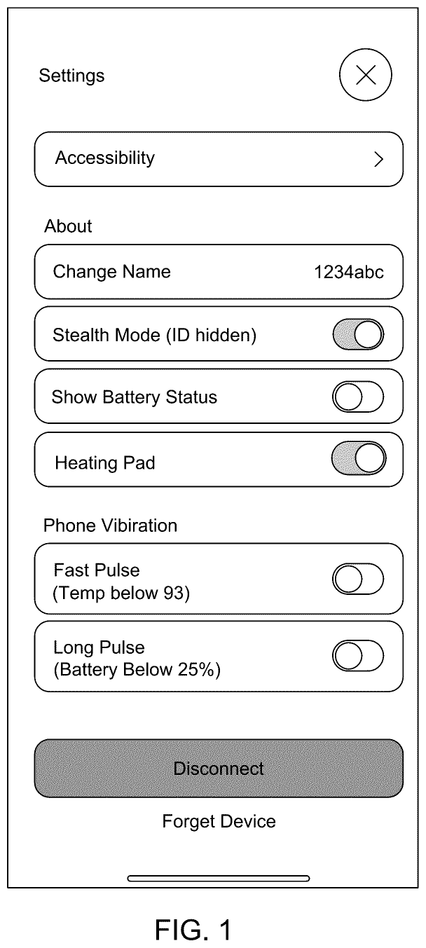

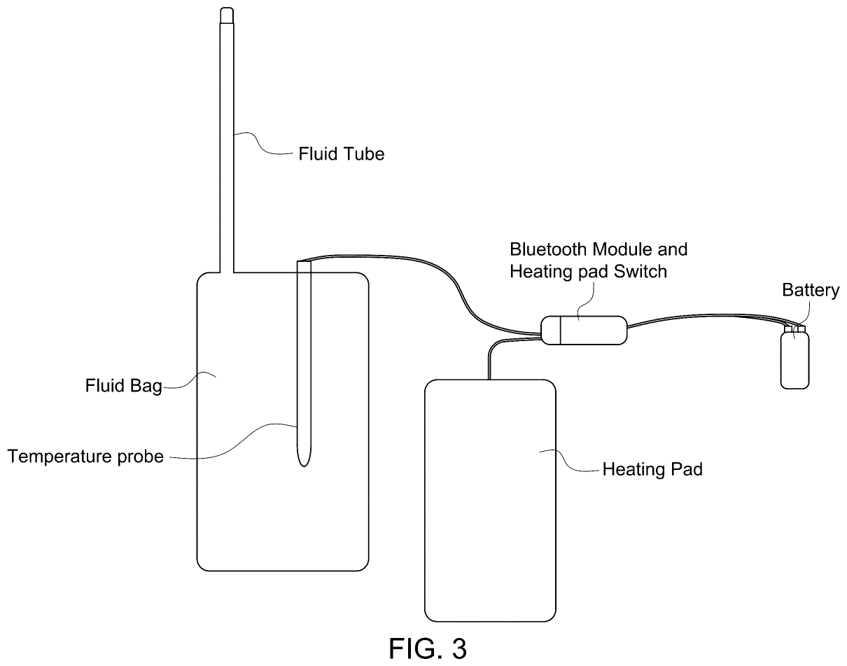

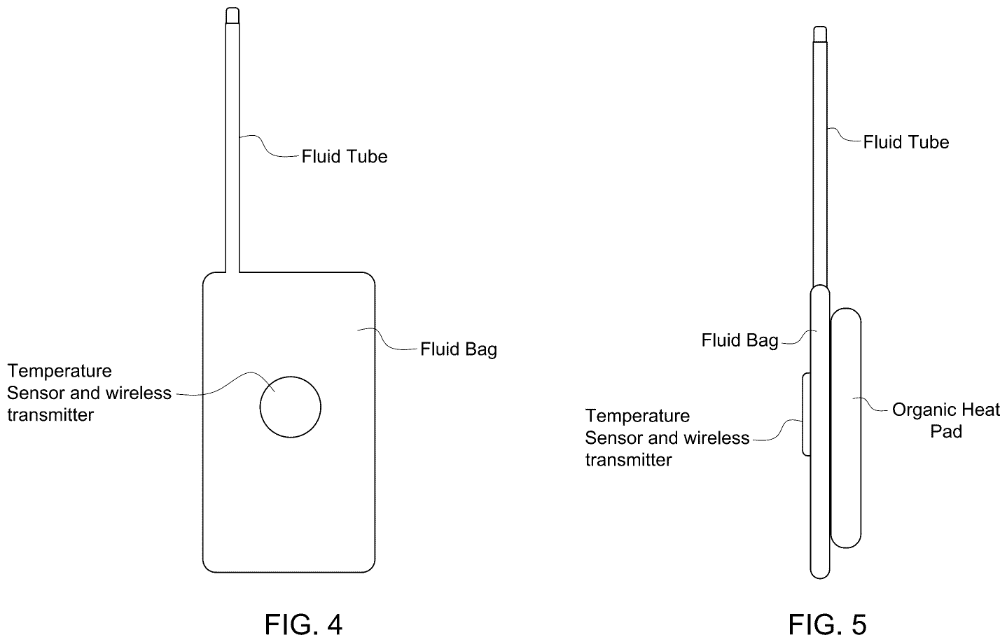

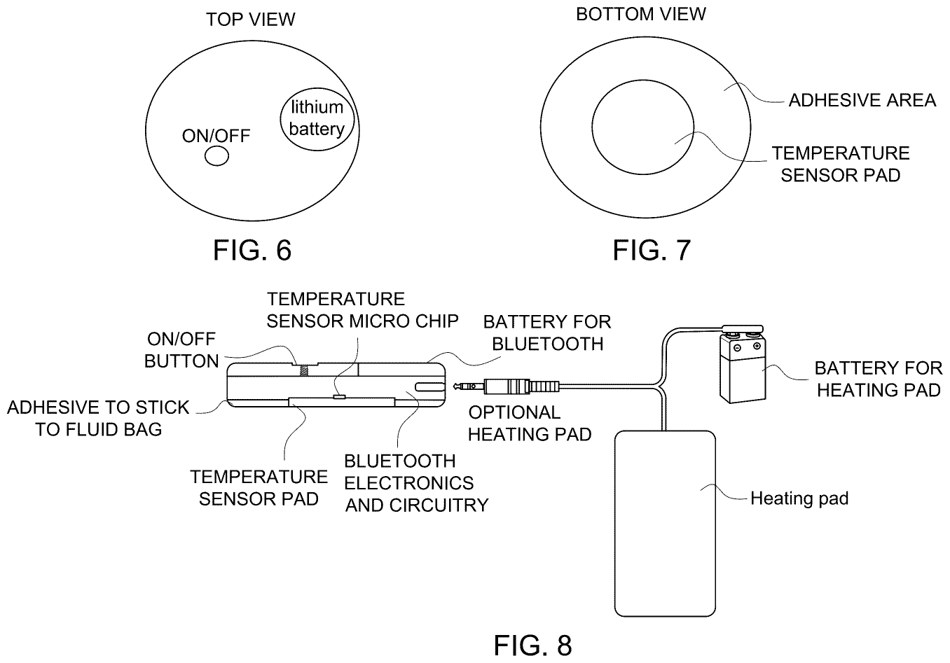

1. A device which monitors and notifies the user of urine temperature comprising of a temperature gauge, heating element, communication method, fluid bag, tube, battery, and phone application.

2. The device which monitors and notifies the user of urine temperature comprising of the temperature gauge, heating element, communication method, fluid bag, tube, battery, and phone application as in claim 1, wherein the temperature gauge allows the phone application to show the current temperature readout regardless of heating source.

3. The device which monitors and notifies the user of urine temperature comprising of the temperature gauge, heating element, communication method, fluid bag, tube, battery, and phone application as in claim 1, wherein the application provides a color-coded range of which corresponds to a digital urine temperature.

4. The device which monitors and notifies the user of urine temperature comprising of the temperature gauge, heating element, communication method, fluid bag, tube, battery, and phone application as in claim 1, wherein the application provides vibration alerts to the user's phone when the temperature of the urine is too low or when battery life is getting low.

5. The device which monitors and notifies the user of urine temperature comprising of the temperature gauge, heating element, communication method, fluid bag, tube, battery, and phone application as in claim 1, wherein the heating element may be activated or deactivated as needed.

6. The device which monitors and notifies the user of urine temperature comprising of the temperature gauge, heating element, communication method, fluid bag, tube, battery, and phone application as in claim 1, wherein the temperature gauge may be molded into a fluid container or adhered to the outside of a container.
